(19) United States
(12) Patent Application Publication
(10) Pub. No.: US 2016/0208214 A1
(43) Pub. Date: Jul. 21, 2016

(54) METHODS AND COMPOSITIONS FOR GENERATION OF DEVELOPMENTALLY-INCOMPETENT EGGS IN RECIPIENTS OF NUCLEAR GENETIC TRANSFER

(71) Applicant: NORTHEASTERN UNIVERSITY, Boston, MA (US)

(72) Inventors: Jonathan L. Tilly, Windham, NH (US); Dori C. Woods, Londonerry, NH (US)

(21) Appl. No.: 15/025,697

(22) PCT Filed: Oct. 2, 2014
(86) PCT No.: PCT/US14/58922

§ 371 Date: Mar. 31, 2016

(51) Int. Cl. C12N 5/075

(52) U.S. Cl. CPC C12N 5/0609; C12N 2510/00; C12N 2517/10

(57) ABSTRACT

The present technology provides for a developmentally-incompetent egg cell that is produced by genetically engineering (e.g., inactivating) at least one gene in an oocyte precursor cell and culturing the oocyte precursor cell in conditions sufficient to produce an egg cell. The present technology also provides for methods of using the developmentally-incompetent egg cell.

### CROSS-REFERENCE TO RELATED APPLICATION

This application claims priority to U.S. Provisional Application No. 61/885,559 filed Oct. 2, 2013, the contents of which are incorporated herein by reference in their entireties.

### GOVERNMENT SUPPORT

The present technology was made with U.S. Government support under grant R37-AG012279 awarded by the National Institutes of Health. The U.S. Government has certain rights in the present technology.

### BACKGROUND

The following description is provided to assist the understanding of the reader. None of the information provided or references cited is admitted to be prior art.

Mitochondria, which provide cellular energy to all cells in the form of adenosine triphosphate (ATP), are critical to successfully fertilization. Maternal (egg)-derived mitochondria serve as the sole source of mitochondria for newly formed embryos, as paternal (sperm)-derived mitochondria are degraded by the egg after fertilization by the sperm. Impaired function of egg mitochondria, which is often observed with advancing maternal age, has been linked to poor embryonic developmental competency that can lead to embryonic growth arrest, embryonic implantation failure, and miscarriage.

Mitochondria are also important in the context of fertility in that disorders rooted in mitochondrial DNA mutations cause a spectrum of human disease, including epilepsy, deafness, diabetes, cardiomyopathy, and liver failure.

### SUMMARY

In one aspect, the present technology relates to a developmentally-incompetent egg cell engineered to express decreased levels, as compared to a wild-type egg cell, of one or more proteins encoded by one or more genes selected from the group consisting of: zygote arrest protein 1 (“ZAR 1”), oocyte secretory protein 1 (“OSP1”), and maternal antigen that embryos require (“MATER”). In some embodiments, the egg cell does not contain detectable levels of the one or more proteins encoded by one or more genes selected from the group consisting of: ZAR 1, OSP1 and MATER.

In some embodiments, the developmentally-incompetent egg cell has been fertilized.

In some embodiments, the developmentally-incompetent egg cell comprises female and male pronuclei.

In some embodiments, the developmentally-incompetent egg cell comprises an inactivated gene selected from the group consisting of ZAR 1, OSP1 and MATER.

In some embodiments, the developmentally-incompetent egg cell has been enucleated.

In another aspect, the present technology relates to a method for producing a developmentally-incompetent egg cell comprising inactivating, in an oocyte precursor cell, one or more genes selected from the group consisting of ZAR 1, OSP1 and MATER; culturing the oocyte precursor cell under conditions to derive the developmentally-incompetent egg cell.

In some embodiments, the oocyte precursor cell is selected from the group consisting of: female germline stem cells, embryonic stem cells, induced pluripotent stem cells, skin cells, bone marrow cells and peripheral blood cells.

In some embodiments, the inactivating comprises one or more techniques selected from the group consisting of: CRISPR/Cas9, transcription activator-like effector nucleases (TALENS), engineered meganucleases, zinc-finger nucleases (ZFNs), site directed mutagenesis, and conditional knockout.

In some embodiments, the method also includes fertilizing the egg cell.

In some embodiments, the method also includes enucleating the developmentally-incompetent egg cell.

In another aspect, the present technology relates to a method for enhancing the mitochondrial health of a donor fertilized egg, comprising introducing the nucleus of the donor fertilized egg into the developmentally-incompetent egg cell described above thereby producing a chimeric donor fertilized egg cell.

In some embodiments, the donor fertilized egg cell carries one or more mitochondrial genetic mutations.

In some embodiments, the donor fertilized egg cell carries a known mitochondrial disease.

In some embodiments, the engineered donor fertilized egg cell undergoes embryogenesis.

In some embodiments, the developmentally-incompetent egg cell is a human egg cell.

### DETAILED DESCRIPTION

It is to be appreciated that certain aspects, modes, embodiments, variations and features of the present technology are described below in various levels of detail in order to provide a substantial understanding of the present technology. The various concepts introduced above and discussed in greater detail below may be implemented in any of numerous ways, as the described concepts are not limited to any particular manner of implementation. Examples of specific implementations and applications are provided primarily for illustrative purposes. The definitions of certain terms as used in this specification are provided below. Unless defined otherwise, all technical and scientific terms used herein generally have the same meaning as commonly understood by one of ordinary skill in the art to which this present technology belongs.

As used herein, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “developmentally-incompetent egg” and “developmentally-incompetent egg cells” are used interchangeably, and refer to an egg cell that is incapable of cleavage and embryogenesis even after fertilization.

### General

Assisted reproductive technology (ART) procedures allow for the transfer of nuclear genetic material (e.g., the nucleus) present in a fertilized egg to be transferred into a fertilized, enucleated egg, i.e., a fertilized egg with the nuclear genetic material removed. An example where such a procedure would be beneficial is in fertilized eggs that are diagnosed with mitochondrial disease. The nuclear genetic material from the mitochondrial diseased fertilized egg, i.e., the donor egg, can be removed and implanted into a fertilized, enucleated egg, i.e., the recipient egg, that expresses healthy mitochondria. The result embryo and offspring would carry the genetic information of the donor egg but would not have the mitochondrial disease.

However, the approach disclosed above does present an ethical hurdle. Since the recipient egg is fertilized prior to enucleation in preparation for receipt of the donor egg's nuclear genetic material, it is unclear if enucleation of the recipient egg results in the sacrifice of a viable embryo. The ethical issues are extremely heightened if such a procedure were to occur in human eggs.

There is no established treatment options currently exist to optimize the energetic potential of eggs and embryos of women undergoing in vitro fertilization (IVF). Also there are no established treatment options to prevent mitochondrial disease inheritance. Mitochondrial disease and disorders can include, but are not limited to, e.g., Alpers Disease, Barth Syndrome, Lethal Infantile Cardiomyopathy (LIC), beta-oxidation defects, carnitine-acyl-carnitine deficiency, carnitine deficiency, creatine deficiency syndromes; co-enzyme Q10 deficiency, Complex I deficiency, Complex II Deficiency, Complex III Deficiency, Complex IV Deficiency/COX Deficiency, Complex V Deficiency, Chronic Progressive External Ophthalmoplegia Syndrome (CPEO), Carnitine palmitoyltransferase (CPT) I Deficiency, CPT II Deficiency; Kearns-Sayre Syndrome (KSS), lactic acidosis, Leukoencephalopathy with brain stem and spinal cord involvement and lactate elevation (BSL—Leukodystrohpy), Long-Chain Acyl-CoA Dehydrongenase Deficiency (LCAD), Long-Chain 3-hydroxyacyl-CoA Dehydrongenase Deficiency (LCHAD); Leigh Disease or Syndrome, Luft Disease, Multiple Acyl-CoA Dehydrogenase Deficiency (MAD/Glutaric Aciduria Type II), Medium-Chain Acyl-CoA Dehydrongenase Deficiency (MCAD), Mitochondrial Encephalomyopathy Lactic Acidosis and Strokelike Episodes (MELAS), Myoclonic Epilepsy and Ragged-Red Fiber Disease (MERRF), Mitochondrial Recessive Ataxia Syndrome (MIRAS), mitochondrial cytopathy, mitochondrial DNA depletion, mitochondrial encephalopathy, mitochondrial myopathy, Myoneurogastointestinal Disorder and Encephalopathy (MNGIE), Neuropathy, Ataxia, and Retinitis Pigmentosa (NARP), Pearson Syndrome, pyruvate carboxylase deficiency, pyruvate dehydrogenase deficiency; POLG Mutations, short-chain acyl-CoA dehydrogenase deficiency (SCAD), short-chain 3-hydroxyacyl-CoA deficiency (SCHAD), and very long-chain acyl-CoA dehydrongenase deficiency (VLCAD).

The present technology provides methods and compositions for producing developmentally-incompetent (“deactivated”) eggs that have no potential to undergo embryogenesis after sperm penetration. Accordingly, these deactivated eggs, which are developmentally incompetent due to a targeted mutation of the existing genetic material are useful in methods for enucleation followed by transfer of genetic material from fertilized eggs of a female with either impaired mitochondrial function (e.g., bioenergetics capacity) or a mitochondrial DNA mutation-based disorder. This approach is advantageous at least because it provides a means to either optimize energetic potential of embryos to improve pregnancy outcomes after IVF, or to prevent mitochondrial disease inheritance, without the ethical issues of potential embryo destruction associated with recipient egg enucleation after sperm penetration (i.e., fertilization).

In one aspect, the present technology provides developmentally-incompetent egg compositions (i.e., deactivate eggs) that cannot undergo embryogenesis after sperm penetration (i.e., fertilization). In another aspect, the present technology provides methods for the preparation of developmentally-incompetent eggs (i.e., deactivate eggs) that cannot undergo embryogenesis after sperm penetration (i.e., fertilization). In another aspect, the present technology provides methods for the use of the developmentally-incompetent eggs.

### Developmentally-Incompetent Egg Compositions of the Present Technology

In one aspect, the present technology provides a developmentally-incompetent egg cell composition. In some embodiments, the developmentally-incompetent egg has reduced level of gene product as compare to a wild type competent egg. In some embodiments, the eggs are developmentally-incompetent as a result of inactivation of at least one gene and comprise at least one inactivated gene. In some embodiments, the inactivation of the at least one gene prevents embryogenesis. In some embodiments, the inactivation of the at least one gene prevents embryogenesis after fertilization. In some embodiments, the inactivated gene or genes results in the prevention of early embryogenesis, mid-embryogenesis, and/or late embryogenesis.

In some embodiments, a developmentally-incompetent egg is a fertilized egg that has at least one inactivated enzyme, wherein the inactivated enzyme prevents embryogenesis of the fertilized egg. In some embodiments, the developmentally-incompetent egg cell comprises female and male pronuclei. Without wishing to be bound by theory, fertilized developmentally-incompetent eggs provide an ethical means for providing an ideal recipient for genetic materials from other fertilized eggs, as fertilized developmentally-incompetent eggs never form a viable embryo; as such enucleating fertilized developmentally-incompetent eggs does not raise ethical issues.

By way of example, but not by way of limitation, in some embodiments, the inactivated gene is a selected from the group consisting of: the zygote arrest protein 1 (ZAR 1) gene, oocyte secretory protein 1 (OSP1) gene, and maternal antigen that embryos require (MATER) gene.

In some embodiments inactivation of one of the genes listed above, results in the subsequent loss of the gene product (e.g., protein), the loss of which prevents the fertilized egg from transitioning to the embryo stage. As such, in some embodiments, the developmentally incompetent egg composition is deficient in the product (i.e., decreased level of the product as compared to wild type) of the inactivated gene.

In some embodiments, the developmentally-incompetent egg is derived from an oocyte precursor cells. By way of example, but not by way of limitation, in some embodiments, the oocyte precursor cells include, but are not limited to, multipotent cell, unipotent cells, female germline stem cells (fGSCs, also known as oogonial stem cells or OSCs), embryonic stem cells (ESCs), induced pluripotent stem cells (iPSCs), bone marrow, peripheral blood, and skin cells.

In some embodiments, the developmentally-incompetent egg is a mammalian egg, a reptilian egg, a fish egg, an amphibian egg, an insect egg, or an avian egg. Mammals from which the egg can originate, include, for example, farm animals, such as sheep, pigs, cows, and horses; pet animals, such as dogs and cats; laboratory animals, such as rats, mice, monkeys, and rabbits. In one embodiment, the mammal is a human.

In some embodiments, the developmentally-incompetent egg does not have a disease. By way of example, but not by way of limitation, disease can include, but is not limited to, a mitochondrial disease or disorder. Mitochondrial disease and disorders can include, but not limited to, e.g., Alpers Disease, Barth Syndrome, Lethal Infantile Cardiomyopathy (LIC), beta-oxidation defects, carnitine-acyl-carnitine deficiency; carnitine deficiency, creatine deficiency syndromes; co-enzyme Q10 deficiency, Complex I deficiency; Complex II Deficiency, Complex III Deficiency, Complex IV Deficiency/COX Deficiency, Complex V Deficiency, Chronic Progressive External Ophthalmoplegia Syndrome (CPEO), Carnitine palmitoyltransferase (CPT) I Deficiency, CPT II Deficiency; Kearns-Sayre Syndrome (KSS), lactic acidosis, Leukoencephalopathy with brain stem and spinal cord involvement and lactate elevation (BSL—Leukodystrohpy), Long-Chain Acyl-CoA Dehydrongenase Deficiency (LCAD), Long-Chain 3-hydroxyacyl-CoA Dehydrongenase Deficiency (LCHAD); Leigh Disease or Syndrome, Luft Disease, Multiple Acyl-CoA Dehydrogenase Deficiency (MAD/Glutaric Aciduria Type II), Medium-Chain Acyl-CoA Dehydrongenase Deficiency (MCAD), Mitochondrial Encephalomyopathy Lactic Acidosis and Strokelike Episodes (MELAS), Myoclonic Epilepsy and Ragged-Red Fiber Disease (MERRF), Mitochondrial Recessive Ataxia Syndrome (MIRAS), mitochondrial cytopathy, mitochondrial DNA depletion, mitochondrial encephalopathy, mitochondrial myopathy, Myoneurogastointestinal Disorder and Encephalopathy (MNGIE), Neuropathy, Ataxia, and Retinitis Pigmentosa (NARP), Pearson Syndrome, pyruvate carboxylase deficiency, pyruvate dehydrogenase deficiency; POLG Mutations, short-chain acyl-CoA dehydrogenase deficiency (SCAD), short-chain 3-hydroxyacyl-CoA deficiency (SCHAD), and very long-chain acyl-CoA dehydrongenase deficiency (VLCAD).

### Preparation of Developmentally-Incompetent Eggs of the Present Technology

In one aspect the present technology provides methods for making developmentally-incompetent eggs.

In some embodiments, a developmentally-incompetent egg is produce by genetically engineering (e.g, inactivating) at least one gene in an oocyte precursor cell, and then culturing the genetically engineered oocyte precursor cell under conditions sufficient to produce developmentally-incompetent egg cells. In some embodiments, the production of the developmentally-incompetent egg cells also includes fertilizing the developmentally-incompetent egg cells.

By way of example, but not by way of limitation, in some embodiments, the precursor cells include, but are not limited to, multipotent cell, unipotent cells, female germline stem cells (fGSCs, also known as oogonial stem cells or OSCs), embryonic stem cells (ESCs), induced pluripotent stem cells (iPSCs), bone marrow, peripheral blood, and skin cells.

In some embodiments, the inactivation of the gene in the oocyte precursor occurs in vitro. In some embodiments, the fertilization of the developmentally-incompetent egg occurs in vitro.

A gene within the oocyte precursor can be inactivated by any method known in the art. By way of example, but not by way of limitation, in some embodiments, a gene within an egg is inactivated by CRISPR/Cas9, transcription activator-like effector nucleases (TALENS), engineered meganucleases, zinc-finger nucleases (ZFNs), site directed mutagenesis, and conditional knockout, e.g., Cre-LoxP system. See, e.g., Sun et al., Biology of Reproduction, 79: 1014-120 (2008).

In some embodiments, the inactivation of at least one gene prevents early embryogenesis in the developmentally-incompetent egg cells. In some embodiments, the inactivation of at least one gene prevents early embryogenesis even after fertilization.

By way of example, but not by way of limitation, in some embodiments, the inactivated gene is selected from the group consisting of: zygote arrest protein 1 (ZAR 1) gene, oocyte secretory protein 1 (OSP1) gene, and maternal antigen that embryos require (MATER) gene.

### Methods of Using Developmentally-Incompetent Eggs

In another aspect, the present technology relates to methods for exchanging nuclear genetic material between two fertilized eggs. In some embodiments, one of the fertilized eggs is initially developmentally-incompetent. In some embodiments, the methods for exchanging nuclear genetic material between two eggs comprise the use of a developmentally-incompetent egg composition of the present technology.

In some embodiments, the method for exchanging genetic material between two eggs comprises:

harvesting a precursor cell;

inactivating at least one gene in the precursor cell;

culturing the precursor cell under condition sufficient to produce a developmentally-incompetent recipient egg;

contacting the developmentally-incompetent recipient egg with sperm in vitro under conditions suitable to produce a fertilized developmentally-incompetent recipient egg;

enucleating the fertilized developmentally-incompetent recipient egg to produce an enucleated developmentally-incompetent recipient egg; and

nucleating the enucleated developmentally-incompetent recipient egg with nuclear genetic materials from a fertilized donor egg under conditions wherein the enucleated developmentally-incompetent recipient egg accepts the nuclear genetic material from the fertilized donor egg to produce a developmentally competent egg. In some embodiments, the nuclear genetic materials comprise a nucleus organelle.

In some embodiments, the method for producing a developmentally-incompetent egg cell includes inactivating, in an oocyte precursor cell, one or more genes selected from the group consisting of ZAR 1, OSP1 and MATER and culturing the oocyte precursor cell under conditions to derive the developmentally-incompetent egg cell.

In some embodiments, the method further comprises adding at least one agent to initiate embryogenesis after nucleating the enucleated developmentally-incompetent recipient egg with the nuclear genetic material from a donor egg. Agents include, but are not limited to, the protein from the inactivated gene, e.g., zygote arrest protein 1, oocyte secretory protein 1, and maternal antigen that embryos require.

In some embodiments, the method for inactivating the gene is one or more techniques selected from the group consisting of: CRISPR/Cas9, transcription activator-like effector nucleases (TALENS), engineered meganucleases, zinc-finger nucleases (ZFNs), site directed mutagenesis, or conditional knockout, e.g., Cre-LoxP system.

By way of example, but not by way of limitation, in some embodiments, the inactivated gene is selected from zygote arrest protein 1 (ZAR 1), oocyte secretory protein 1 (OSP1), and maternal antigen that embryos require (MATER). In some embodiments, inactivation of at least one gene prevents embryogenesis after fertilization of the egg. In some embodiments, the inactivation of the gene prevents early, mid, or late embryogenesis.

In some embodiments, the inactivation of the gene is performed ex vivo or in vitro.

In some embodiments, the recipient egg does not have a disease. In some embodiments, the recipient egg does not have a mitochondrial disease.

In some embodiments, the fertilized donor egg has a disease or disorder. In some embodiments, the disease is selected from the group consisting of: Alpers Disease, Barth Syndrome, Lethal Infantile Cardiomyopathy (LIC), beta-oxidation defects, carnitine-acyl-carnitine deficiency, carnitine deficiency, creatine deficiency syndromes; co-enzyme Q10 deficiency, Complex I deficiency, Complex II Deficiency, Complex III Deficiency, Complex IV Deficiency/COX Deficiency, Complex V Deficiency, Chronic Progressive External Ophthalmoplegia Syndrome (CPEO), Carnitine palmitoyltransferase (CPT) I Deficiency, CPT II Deficiency; Kearns-Sayre Syndrome (KSS), lactic acidosis, Leukoencephalopathy with brain stem and spinal cord involvement and lactate elevation (BSL—Leukodystrohpy), Long-Chain Acyl-CoA Dehydrongenase Deficiency (LCAD), Long-Chain 3-hydroxyacyl-CoA Dehydrongenase Deficiency (LCHAD); Leigh Disease or Syndrome, Luft Disease, Multiple Acyl-CoA Dehydrogenase Deficiency (MAD/Glutaric Aciduria Type II), Medium-Chain Acyl-CoA Dehydrongenase Deficiency (MCAD), Mitochondrial Encephalomyopathy Lactic Acidosis and Strokelike Episodes (MELAS), Myoclonic Epilepsy and Ragged-Red Fiber Disease (MERRF), Mitochondrial Recessive Ataxia Syndrome (MIRAS), mitochondrial cytopathy, mitochondrial DNA depletion, mitochondrial encephalopathy, mitochondrial myopathy, Myoneurogastointestinal Disorder and Encephalopathy (MNGIE), Neuropathy, Ataxia, and Retinitis Pigmentosa (NARP), Pearson Syndrome, pyruvate carboxylase deficiency, pyruvate dehydrogenase deficiency; POLG Mutations, short-chain acyl-CoA dehydrogenase deficiency (SCAD), short-chain 3-hydroxyacyl-CoA deficiency (SCHAD), and very long-chain acyl-CoA dehydrongenase deficiency (VLCAD).

In some embodiments, the developmentally-incompetent egg cell with the nuclear genetic material from a donor egg is useful for improving fertility.

In some embodiments, the developmentally-incompetent egg cell with the nuclear genetic material from a donor egg is useful for reducing the inheritance of genetic diseases, e.g., mitochondrial diseases or disorders.

In some embodiments, the developmentally-incompetent egg cell with the nuclear genetic material from a donor egg is useful for enhancing the mitochondrial health of a donor fertilized egg cell, wherein introducing the nucleus of the donor fertilized egg cell into the developmentally-incompetent egg cell produces an engineered healthy donor fertilized egg cell.

In some embodiments, developmentally-incompetent egg cells with the nuclear genetic material from a donor egg are useful as an option to optimize the energetic potential of eggs and embryos of women undergoing in vitro fertilization.

In some embodiments, developmentally-incompetent egg cells with the nuclear genetic material from a donor egg are useful as treatment option to prevent mitochondrial disease.

### Kits

In some embodiments, the present technology relates to kits. In some embodiments, the kit includes at least one fertilized developmentally-incompetent egg of the present technology and instructions for its use in the methods of the present technology.

In some embodiments, the kit also includes tools for enucleation and/or nucleation. Additionally, or alternatively, in some embodiments, the kit also includes solutions for storing and/or enucleating or nucleating the fertilized egg.

In some embodiments, the fertilized developmentally-incompetent egg of the present technology does not have a mitochondrial disease. In some embodiments, the fertilized developmentally-incompetent egg of the present technology does not have a disease.

In some embodiments, the fertilized developmentally-incompetent egg of the present technology is mammalian egg, a reptilian egg, a fish egg, an amphibian egg, an insect egg, or an avian egg. Mammals from which the egg can originate, include, for example, farm animals, such as sheep, pigs, cows, and horses; pet animals, such as dogs and cats; laboratory animals, such as rats, mice, monkeys, and rabbits. In one embodiment, the mammal is a human.

In some embodiments, the kit also includes instructions for how to use the kit. By way of example, but not by limitation, in some embodiments, the instructions would disclose how to enucleate the fertilized egg in the kit and how to enucleate and then nucleate the enucleated fertilized developmentally-incompetent egg of the present technology with nuclear genetic material from another fertilized egg.

### EXAMPLES

The present examples are non-limiting implementations of the use of the present technology.

### Example 1

### TALENs Knockout of ZAR 1 Prevents Embryogenesis in Mice

This example shows that knockout of ZAR 1 in mice oocytes prevents embryogenesis in the ZAR 1 KO oocytes after in vitro fertilization (IVF).

### Materials and Methods

TALEN: TALEN protein is an artificial sequence-specific endonuclease that contains Xanthomonas transcription activator-like effector (TALE) and a nuclease domain of FokI restriction endonuclease. DNA binding domain of TALE consists of a tandem repeat of 33-35 amino acid motifs in which there are two critical adjacent amino acid pairs called a repeat variable diresidue (RVD) that determines the binding specificity for single nucleotide. There is a one-to-one relationship between the RVD and its recognition nucleotide. Using this code, a TALEN can be constructed with a DNA binding motif recognizing the desired nucleotide sequence. When two TALENs are expressed in a cell and bind to the genome at an appropriate distance, called a spacer, the nuclease domain of FokI dimerizes and generates a double-strand break (DSB) within the spacer. The lesion is frequently repaired via nonhomologous end joining (NHEJ), an error-prone mechanism that results in the introduction of small insertion or deletion (indel) mutations. It has been reported that TALENs are useful for creating KO animals, such as fruit flies, silkworms, zebra fish, Xenopus and rats. See, e.g., Kato et al., Production of Sry knockout mouse using TALEN via oocyte injection, SCIENTIFIC REPORTS (Nov. 5, 2013).

The TALEN plasmids are designed for ZAR 1 using the online TAL Effector Nucleotide Targeter 2.0 software program. The TALENs are assembled in pcDNA-TAL-NC vector plasmids. Vector plasmids with a control vector are used as controls.

Microinjection: TALEN plasmids and control plasmids are digested by PvuII restriction endonuclease. One microgram of each digested plasmids is used as a template for the in vitro transcription reaction using the mMESSAGE mMACHINE T7 Kit (Life Technologies) according to the manufacturer's instructions. The synthesized RNAs are purified using the MegaClear kit (Life Technologies) according to the manufacturer's instructions. The RNA concentration are determined using a NanoDrop 1000 spectrophotometer and diluted with injection buffer (10 mM Tris-HCl/0.1 mM EDTA (pH 7.4)) at 600 ng/ml, wherein there is a total of two TALEN mRNAs (1:1 ratio, i.e., 300 ng/ml each). The microinjection of the two TALEN mRNAs mix and control vector into cytoplasm of oocytes is carried out under standard procedures using oocytes obtained from superovulated (C57BL/6 3 DBA2) F1 mice.

In vitro fertilization: A concentration of about 2×10⁵ sperm cells/ml in potassium simplex optimized medium (KSOM) supplemented with 4 mg/ml BSA was prepared, and conventional IVF is performed and embryogenesis is measured.

### Results

It is anticipated that the oocytes treated with the TALENs designed for ZAR 1 will not display significant signs of embryogenesis after IVF as compared to the oocytes treated with the control vector. These results will show that it is useful to inactivate specific genes to produce developmentally-incompetent eggs.

### Example 2

### Mouse Embryonic Stems Cells Engineered to Produce Oocyte Precursor Cell with a Knock Out ZAR 1 Gene

Using the method described above, embryonic stems cells are engineered to have an inactive ZAR 1 gene. The ZAR 1 deficient stem cells are cultured under conditions to differentiate into developmentally-incompetent eggs, e.g., ZAR 1 deficient egg cells.

The developmentally-incompetent eggs are subjected to in vitro fertilization. In vitro fertilized wild type mouse eggs are used as a control.

It is anticipated that the developmentally-incompetent eggs derived from mouse embryonic stem cells will form female and male pronuclei after fertilization. However, the developmentally-incompetent eggs will not cleave or enter embryogenesis as compared to the control cell.

These results will show that pluripotent stem cell can be used to generate developmentally-incompetent eggs.

### Example 3

### Nuclear Genetic Material Transfer from Wild-Type Fertilized Egg Recovers ZAR 1 Knockout Inhibition of Embryogenesis

This example shows transfer of nuclear genetic materials from a wild-type fertilized egg will recover ZAR 1 knockout inhibition of embryogenesis.

### Materials and Methods

Fertilized ZAR 1 knockout eggs are generated using the protocol described in Example 1 or 2.

Eggs from a wild-type female mouse are harvested and fertilized by the in vitro fertilization protocol discussed above. Eggs from a wild-type female mouse not subject to in vitro fertilization are used as controls.

The ZAR 1 knockout eggs produced by Examples 1 or 2 are enucleated to remove their nuclear genetic materials. The enucleated ZAR 1 knockout eggs are re-nucleated with the nucleus from a fertilized wild type egg or the nucleus from an unfertilized wild type egg. The re-nucleated eggs are tested for embryogenesis and normal development after 1 day, 2 days, 3 days, 4 days, 5 days, 6 days, 7 days, and/or 2 weeks after the re-nucleation.

### Results

It is anticipated that the ZAR 1 knockout eggs re-nucleated with the nuclear genetic material from a fertilized wild type egg will exhibit embryogenesis and signs of normal development as compared to the control (i.e., the ZAR 1 knockout eggs re-nucleated with the nuclear genetic material from an unfertilized wild type egg).

These results will show that developmentally-incompetent eggs, i.e., ZAR 1 knockout eggs, can be rescued into developmentally-competent eggs by transfer of nuclear genetic materials from a wild type fertilized egg. Accordingly, developmentally-incompetent eggs of the present technology are useful as recipients for nuclear genetic material from other eggs as they can become embryonically competent with the nucleation of embryonically active nuclear genetic materials.

### Example 4

### Transfer of Nuclear Genetic Material from Mitochondrial Diseased Donor Egg into ZAR 1 Knockout Developmentally-Incompetent Recipient Egg

This example shows that developmentally-incompetent eggs can serve as recipients of nuclear genetic materials from mitochondrial diseased donor eggs and develop into a normal embryo, i.e., does not exhibits signs of mitochondrial dysfunction or disease.

### Materials and Methods

Fertilized ZAR 1 knockout eggs are generated using the protocol described in Example 1 or 2.

Eggs from a mitochondrial disease female mouse are harvested and fertilized by the in vitro fertilization protocol discussed above.

The fertilized ZAR 1 knockout eggs are enucleated to remove their nuclear genetic materials. The enucleated ZAR 1 knockout eggs are re-nucleated with the nucleus from a fertilized mitochondrial diseased egg. Fertilized eggs from a mitochondrial disease female mouse not subject nuclear genetic material transfer into are used as controls. The re-nucleated eggs and control eggs are tested for embryogenesis and normal development after 1 day, 2 days, 3 days, 4 days, 5 days, 6 days, 7 days, and/or 2 weeks after the re-nucleation.

### Results

It is anticipated that the ZAR 1 knockout eggs re-nucleated with the nucleus from the fertilized mitochondrial diseased eggs will exhibit improved embryogenesis and normal development as compared to the fertilized mitochondrial diseased control eggs. These results will show that the developmentally-incompetent eggs can rescue normal development of mitochondrial diseased eggs. Accordingly, the developmentally-incompetent eggs of the present technology are useful in preventing the transmission of mitochondrial disease on to offspring.

### EQUIVALENTS

The present technology is not to be limited in terms of the particular embodiments described in this application, which are intended as single illustrations of individual aspects of the present technology. Many modifications and variations of the present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

1. A developmentally-incompetent egg cell engineered to express decreased levels, as compared to a wild-type egg cell, of one or more proteins encoded by one or more genes selected from the group consisting of: zygote arrest protein 1 (“ZAR 1”), oocyte secretory protein 1 (“OSP 1”), and maternal antigen that embryos require (“MATER”).

2. The developmentally-incompetent egg cell of claim 1, wherein the egg cell does not contain detectable levels of the one or more proteins encoded by one or more genes selected from the group consisting of: ZAR 1, OSP 1 and MATER.

3. The developmentally-incompetent egg cell of claim 1, that has been fertilized.

4. The developmentally-incompetent egg cell of claim 1, comprising female and male pronuclei.

5. The developmentally-incompetent egg cell of claim 1, comprising an inactivated gene selected from the group consisting of ZAR 1, OSP 1 and MATER.

6. The developmentally-incompetent egg cell of claim 1 that has been enucleated.

7. A method for producing a developmentally-incompetent egg cell, comprising:
inactivating, in an oocyte precursor cell, one or more genes selected from the group consisting of ZAR 1, OSP 1 and MATER; and
culturing the oocyte precursor cell under conditions to derive the developmentally-incompetent egg cell.

8. The method of claim 7, wherein the oocyte precursor cell is selected from the group consisting of: female germline stem cells, embryonic stem cells, induced pluripotent stem cells, skin cells, bone marrow cells and peripheral blood cells.

9. The method of claim 7, wherein inactivating comprises one or more techniques selected from the group consisting of: CRISPR/Cas9, transcription activator-like effector nucleases (TALENS), engineered meganucleases, zinc-finger nucleases (ZFNs), site directed mutagenesis, and conditional knockout.

10. The method of claim 7, further comprising fertilizing the developmentally-incompetent egg cell.

11. The method of claim 7, further comprising enucleating the developmentally-incompetent egg cell.

12. A method for enhancing the mitochondrial health of a donor fertilized egg cell, comprising:
introducing the nucleus of the donor fertilized egg cell into the developmentally-incompetent egg cell of claim 6, thereby producing an engineered donor fertilized egg cell.

13. The method of claim 12, wherein the donor fertilized egg cell carries one or more mitochondrial genetic mutations.

14. The method of claim 12, wherein the donor fertilized egg cell carries a known mitochondrial disease.

15. The method of claim 12, wherein the engineered donor fertilized egg cell undergoes embryogenesis.

16. The method of claim 7, wherein the developmentally-incompetent egg cell is a human egg cell.

17. A kit comprising the developmentally-incompetent egg cell of claim 1; and instructions for using the kit.

18. The kit of claim 17, wherein the developmentally-incompetent egg cell is a human egg cell.

19. The method of claim 12, wherein the developmentally-incompetent egg cell is a human egg cell.

20. The developmentally-incompetent egg cell of claim 1, wherein the developmentally-incompetent egg cell is a human egg cell.
